(19) United States
(12) Patent Application Publication
(10) Pub. No.: US 2024/0285473 A1
(43) Pub. Date: Aug. 29, 2024

(54) SMART PILL DISPENSER PRESCRIPTION TREATMENT SYSTEM

(71) Applicant: 10xBeta, Brooklyn, NY (US)

(72) Inventor: Frederick Zacharias KRUGER, Brooklyn, NY (US)

(73) Assignee: 10xBeta, Brooklyn, NY (US)

(21) Appl. No.: 18/574,105

(22) PCT Filed: Jul. 6, 2022
(86) PCT No.: PCT/US2022/036173

§ 371 Date: Dec. 26, 2023

(51) Int. Cl. A61J 7/00; A61J 1/14; A61J 7/04

(52) U.S. Cl. CPC A61J 7/0076; A61J 1/1468; A61J 7/0481; A61J 2200/70

(57) ABSTRACT

A securable and trackable pill dispenser assembly, a system for tracking and administering medication with the pill dispenser assembly, and related methods are presented herein. The pill dispenser assembly can include a tamper resistant cartridge and a smart pill dispensing portion. The pill dispensing portion of the pill dispenser assembly can be configured to authenticate a patient to the pill dispenser assembly and dispense medication according to a prescription accessible to the dispenser portion. The patient may be able to separate the cartridge from the dispenser portion at the end of a prescription period and mate a new cartridge to the dispenser portion for a new prescription period. The cartridge can be filled by a dispensing pharmacy and/or a pharmaceutical partner and delivered to the patient. The cartridge can inhibit extraction of medication when not mated to the dispensing portion.

### CROSS-REFERENCE TO RELATED APPLICATIONS

This application claims priority to U.S. Provisional Patent Application No. 63/218,704 filed on Jul. 6, 2021, the disclosure of which is incorporated herein by reference in its entirety.

### FIELD

The present invention generally relates to systems, devices, and methods for managing prescription treatments, and more specifically to treatments involving adherence compliance to pill form medication.

### BACKGROUND

The challenge of ensuring safe prescription drug use at home is significant. Each year in the United States 153 million opioid prescriptions are written (according to 2019 reports from the United States Center for Disease Control, CDC), each posing a risk of accidental or deliberate misuse, by patients, children, and others, with the potential to contribute to the national opioid crisis. More generally, correct medication management poses challenges to young and old alike, with the CDC reporting 1.3 million emergency department (ED) visits per year for adverse drug events. This includes approximately 200,000 visits involving children under 17, a subset of approximately 50,000 which are due to unsupervised ingestions by children under 5, and approximately 450,000 adults over 65, who often struggle with complicated regimes.

Many attempted solutions to prevent accidental and intentional drug misuse exist from simple pill boxes, to active reminder caps, to apps, to packaged sachets, to regulatory approaches, however improved solutions are desired.

### SUMMARY

A securable and trackable pill dispenser assembly, system for tracking and administering medication with the pill dispenser assembly, and related methods are presented herein. The pill dispenser assembly can include a tamper resistant cartridge and a smart pill dispensing portion. The pill dispensing portion of the pill dispenser assembly can be configured to authenticate a patient to the pill dispenser assembly and dispense medication according to a prescription uploaded to the dispenser portion and/or otherwise accessible to the dispenser portion. The patient may be able to separate the cartridge from the dispenser portion at the end of a prescription period and mate a new cartridge to the dispenser portion for a new prescription period. The cartridge can be filled by a dispensing pharmacy and/or a pharmaceutical partner and delivered to the patient. The cartridge inhibit extraction of medication when not mated to the dispensing portion. Optionally, the patient can return used cartridges with unused medication to a recycling/disposal center which can then dispose of the unused medication and/or send the cartridge to a pill dispenser distribution center for additional uses.

An example pill dispenser assembly can include a dispenser portion and a cartridge. The dispenser portion can include a dispensing cavity, a movable cavity configured to move to move a medication pill into the dispensing cavity, a processor, and non-transitory computer readable memory in communication with the processor. The cartridge can include a shell portion and an angled barrier extending within the shell portion. The shell portion and the angled barrier can bound a primary cavity of the cartridge that is sized to receive a plurality of medication pills. The cartridge can be configured to couple to the dispenser portion. The cartridge can be configured to secure the plurality of medication pills from removal from the primary cavity when the cartridge is decoupled from the dispenser portion. The non-transitory computer readable memory can include instructions thereon, that when the cartridge is coupled to the dispenser portion and the instructions are executed by the processor, the instructions cause the pill dispenser assembly to authenticate a user of the pill dispenser assembly and actuate, at least in part in response to authentication of the user, the dispenser portion to move the movable cavity and dispense the medication pill into the dispensing cavity such that the medication pill is removable from the pill dispenser assembly.

The dispenser portion can further include a mating protrusion configured to abut the angled barrier of the cartridge when the cartridge is coupled to the dispenser portion.

The dispenser portion can further comprise a biometric sensor. The dispenser portion can be configured to actuate to dispense the medication pill in response to an authentication signal provided from the biometric sensor.

The dispenser portion can further include a central opening in the mating protrusion. The cartridge can further include a central tube extending within the shell such that when the mating protrusion is inserted into the opening of the shell portion the central opening and the central tube are aligned on a longitudinal axis of the pill dispenser assembly.

The mating protrusion can further include an off-axis opening that is positioned radially offset from a longitudinal axis of the pill dispenser assembly. The dispenser portion can further include a pill passage sized to allow the medication pill to pass into the off-axis opening, through the pill passage, and out of the pill dispensing cavity.

The dispenser portion can further include a rechargeable battery and inductive charger.

The dispenser portion can further include a status indicator configured to provide a visual indication of a status of the pill dispenser assembly.

The status indicator can further include a color-changing light ring circumscribing the dispenser portion.

The instructions on the non-transitory computer readable medium can further include prescription information of the user. The processor can be configured to actuate the dispenser portion to dispenser the medication pill based at least in part on the prescription information.

The dispenser portion can further include communication circuitry configured to communicate to an external computing device external to the pill dispenser assembly. When the instructions are executed by the processor, the processor can further receive the prescription information from the external computing device.

The angled barrier can include disks each with a respective opening such that at least one of the disks is rotatable to move the openings into alignment and rotatable to move the openings out of alignment. The angled barrier can further include an opening through which a portion of the mating protrusion is sized to extend. The dispensing portion can include a motor configured to cause the portion of the mating protrusion to rotate and thereby cause the at least one of the disks to rotate to move the openings into alignment and/or to move the openings out of alignment.

An example user device can include a user interface, one or more transceivers, one or more processors, and non-transitory computer readable medium with instructions thereon. The instructions can be executed by the processor to cause the user device to communicate, via the one or more transceivers, to a pill dispenser and display, on the user interface, a dosing schedule associated with the pill dispenser.

The instructions can be executed by the processor to further cause the user device to onboard a cartridge of the pill dispenser.

The instructions can be executed by the processor to further cause the user device to communicate, via the one or more transceivers, to a dose manager configured to provide an interface to a physician. The instructions can be executed by the processor to further cause the user device to receive, via the one or more transceivers, from the dose manager, a prescription adjustment notification. The instructions can be executed by the processor to further cause the user device to transmit, via the one or more transceivers, to the pill dispenser, prescription adjustment instructions based at least in part on the prescription adjustment notification.

The instructions can be executed by the processor to further cause the user device to communicate, via the one or more transceivers, to a dispenser manager configured to provide an interface to a pill dispenser manager. The instructions can be executed by the processor to further cause the user device to receive, via the one or more transceivers, from the dispenser manager, an initial configuration of the pill dispenser. The instructions can be executed by the processor to further cause the user device to transmit, via the one or more transceivers, to the pill dispenser, configuration instructions based at least in part on the initial configuration.

The instructions can be executed by the processor to further cause the user device to transmit, via the one or more transceivers, to the dispenser manager, data related to a life cycle of the pill dispenser.

An example dose manager one or more transceivers, one or more processors, and non-transitory computer readable medium with instructions thereon that can be executed by the processor. The instructions can be executed by the processor to further cause the user device to communicate, via the one or more transceivers, to a pill dispenser. The instructions can be executed by the processor to further cause the user device to receive, via the one or more transceivers, from the pill dispenser, patient compliance information. The instructions can be executed by the processor to further cause the user device to transmit, via the one or more transceivers, to the pill dispenser, prescription information. The does manager can further include a user interface configured to receive prescription information from a health care provider such as a physician and/or display the patient compliance information to the health care provider. The example dose manager can be realized as a HIPPA compliant web portal.

An example dispenser manager can include one or more transceivers, one or more processors, and non-transitory computer readable medium with instructions thereon that can be executed by the processor. The instructions can be executed by the processor to cause the user device to communicate, via the one or more transceivers, to a pill dispenser. The instructions can be executed by the processor to further cause the user device to receive, via the one or more transceivers, from the pill dispenser, data related to a life cycle of the pill dispenser. The instructions can be executed by the processor to further cause the user device to transmit, via the one or more transceivers, to the pill dispenser, an initial configuration of the pill dispenser. The dispenser manager can further include a user interface configured to communicate with a pharmacy or other party responsible for filling prescriptions and/or monitoring pill dispenser functionalities.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is an illustration of components of an example pill dispenser assembly according to aspects of the present invention.

FIG. 1B is an illustration of the example pill dispenser assembly of FIG. 1A in which a cartridge is affixed to a dispenser portion of the example pill dispenser assembly according to aspects of the present invention.

FIG. 1C is an illustration of an example cartridge of the example pill dispenser assembly of FIGS. 1A and 1B in which the cartridge secures medication according to aspects of the present invention.

FIG. 2 is an illustration of a first medication distribution model to provide medication to a new patient according to aspects of the present invention.

FIG. 3 is an illustration of a second medication distribution model to provide medication to an existing patient according to aspects of the present invention.

FIG. 4 is an illustration of a third medication distribution model to provide medication to a new patient according to aspects of the present invention.

FIG. 5 is an illustration of a fourth medication distribution model to provide medication to an existing patient according to aspects of the present invention.

FIG. 6 is an illustration of a prescription treatment system according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal. Computer readable medium can be non-transitory. Non-transitory computer-readable media include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable instructions and/or data.

As used herein, the term “computing system” is intended to include stand-alone machines or devices and/or a combination of machines, components, modules, systems, servers, processors, memory, detectors, user interfaces, computing device interfaces, network interfaces, hardware elements, software elements, firmware elements, and other computer-related units. By way of example, but not limitation, a computing system can include one or more of a general-purpose computer, a special-purpose computer, a processor, a portable electronic device, a portable electronic medical instrument, a stationary or semi-stationary electronic medical instrument, or other electronic data processing apparatus.

As used herein, the term “non-transitory computer-readable media” includes, but is not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Developed based on sophisticated user-centered design studies, some example systems presented herein are configured to provide medication management covering dispensing, security, dosing, adherence, tracking, and disposal. Some examples presented herein include an internet of things (IoT) enabled, ergonomic dispensing device assembly having a secure pill cartridge, a fingerprint-based biometric security, and a unique mechanical design that adjusts to a wide variety of pill sizes and dispenses only one pill at a time. The dispensing device assembly can incorporate mechanical and sensing defenses to tampering. The dispensing device assembly can include a dispensing portion and a cartridge. The dispensing portion can be paired with a smartphone application that onboards new cartridges, verifies dosing schedules and allows for dynamic prescription adjustment and titration, as specified by the provider. Some example medication management systems can be configured to maintain continuous accounting for the medication cartridge, medication therein, and the dispensing portion of the dispensing device assembly. A (Health Insurance Portability and Accountability Act) HIPPA compliant web portal can allow doctors to manage patients and prescriptions, view patient compliance in real-time, and initiate actions. By collecting and aggregating data from many patients the management portal also provides the opportunity to facilitate drug use and efficacy studies. When a treatment regime is complete the system locks and prompts for cartridge return to a central reclamation location for safe disposal of unused medication.

To illustrate potential use cases of various aspects of the present invention, three hypothetical use scenarios are presented.

In the first hypothetical use scenario, a teenage football player is prescribed Percocet with instructions to take 1 or 2 tablets every 6 hours as needed. An example pill management system including a dispensing device assembly tracks his dosing and on Day 2 asks if he wants to reduce to 1 pills. On Day 3 it suggests transitioning to an OTC painkiller. On day 5 the system sends a report to his doctor and during a follow up call the athlete confirms that he is doing well, the doctor initiates a device lock, and the system automatically sends out a FedEx box and return label to retrieve the unused pills.

In the second hypothetical use scenario, it is 3 AM and two parents are awake trying to remember who gave their 5-year-old her last dose of Amoxicillin for her ear infection. One glance at the application on one more user devices of the parents will confirm that one parent gave their daughter her last dose at 8 PM and it will be the other parent's turn to give Amoxicillin to their daughter at 8 AM.

In the third hypothetical use scenario, a 75 year old man is prescribed 0.5 mg Warfarin tablets with the daily dosing is adjusted based on coagulation tests. His doctors review his latest test and push an adjustment from 3 to 2 pills per day to his dispensing device assembly. The next morning the dispensing device assembly activates three times to dispense three tablets.

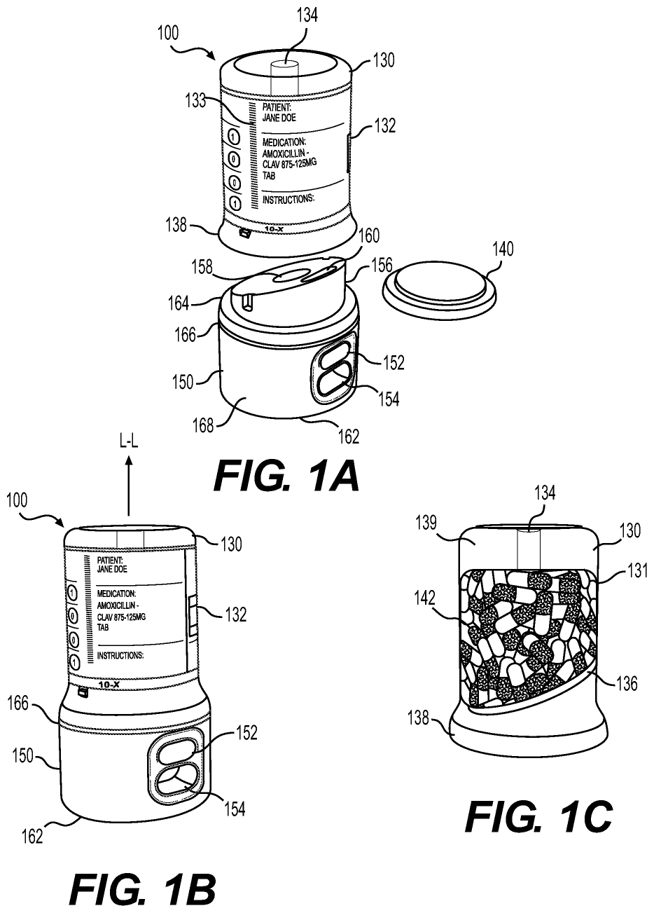

FIG. 1A is an illustration of components of an example pill dispenser assembly 100. The pill dispenser assembly 100 includes a cartridge 130 and a dispenser portion 150. The dispenser portion 150 can include electronic circuitry including a processor, memory, sensors, a power source, communication circuitry for communicating to one or more external computing devices, and other such components to facilitate various functions of the pill dispenser assembly 100 as understood by a person skilled in the pertinent art according to the teachings herein. In some examples, a bottom surface 162 of the dispenser portion 150 can be configured for inductive charging of a rechargeable battery within the dispenser portion 150. Alternatively, the dispenser portion 150 can be configured for wired charging. The cartridge 130 can be purely mechanical, lacking electronics. Alternatively, the cartridge 130 can include circuitry (e.g. radio frequency identification “RFID” transmitter) configured to communicate with the dispenser portion 150 to provide prescription information to the dispenser portion 150. The dispenser portion 150 can further include electronic circuitry to perform payment transactions, e.g. include software and hardware to store credit card information associated with a health savings or reimbursement account such that the dispenser portion 150 can be tapped to a credit card reader at the pharmacy to pay for prescriptions.

The pill dispenser assembly 100 can further include a cartridge cover 140 which can be placed over a mating end 138 of the cartridge 130 to provide protection against mechanical damage to the mating end 138 when the cartridge 130 is shipped and/or handled while decoupled from the dispenser portion 150. The cartridge 130 can be sized, shaped, and otherwise configured to receive a label 132 indicating prescription information such as medication, dosage, number of refills, warnings, etc. The label 132 can be color coded to indicate risk of medication (e.g. green=low risk, yellow=medium risk, red=high risk).

A mating protrusion 156 extending from a top surface 164 of the dispenser portion 150 can be inserted into the mating end 138 of the cartridge 130. The mating protrusion 156 can include a central opening 158 aligned with a central tube 134 of the cartridge 130 and an off-axis opening 160. The dispenser portion 150 can be configured to obtain prescription information from the cartridge 130 prior to and/or upon mating the dispenser portion 150 to the cartridge 130. The cartridge 130 can include an identification (ID) tag 133 that can be observed by a mating sensor of the dispenser portion 150. As illustrated, the ID tag 133 includes a bar code, although other suitable identifying graphic such as a quick response “QR” code and the like may be alternatively or additionally included. The dispenser portion 150 can include a camera positioned together with a biometric sensor 152 or elsewhere on the dispenser portion 150 positioned to view the ID tag 133 prior to and/or after the dispenser portion 150 and the cartridge 130 are mated. In addition, or as an alternative to the visual ID tag 133 and dispenser camera, the ID tag 133 can include an electronic component and the dispenser portion can include an electronic ID sensor (disposed at least in part on the surface of the dispenser portion or entirely internal to the dispenser portion) that is configured to sense the electronic component of the ID tag 133. For instance, the ID tag 133 can include an RFID transmitter and the dispenser portion can include an RFID receiver.

The cartridge 130 and the dispensing portion 150 can include a locking system that can be actuated to secure the cartridge 130 to the dispensing portion 150 when the mating protrusion 156 is inserted into the mating end 138 of the cartridge 130. The locking system can include mechanical features that move upon electrical activation, i.e. electromechanical locks. Preferably, the dispensing portion 150 includes electrical circuitry configured to activate the electromechanical locks upon insertion of the mating protrusion 156 of the dispenser portion 150 into the mating end 138 of the cartridge 130.

FIG. 1B is an illustration of the example pill dispenser assembly 100 which the cartridge 130 is affixed to a dispenser portion 150. Referring collectively to FIGS. 1A and 1B, the central tube 134 of the cartridge 130 and the central opening 158 of the dispenser portion 150 can be aligned on a longitudinal axis L-L of the dispenser assembly 100 when the dispenser assembly 100 is assembled. The dispenser portion 150 includes a pill dispensing cavity 154 sized, shaped, and otherwise configured to allow passage of medication from the dispenser portion 150 when the pill dispenser assembly 100 is assembled and activated to dispense medication. The dispenser portion 150 can include a pill passageway such that a medication pill can enter the pill passageway through the off-axis opening 160, travel through the pill passageway, and exit the dispenser portion 150 through the pill dispensing cavity 154. The dispenser portion 150 can include a rotatable chamber contained within the dispenser portion (not illustrated) that is configured to receive one medication pill at a time and is configured to rotate to release the pill through the dispensing cavity 154. The dispenser portion 150 can include a sensor to verify that the rotatable portion of the dispenser portion includes the medication pill, and the circuitry of the dispenser portion 150 can be configured to rotate the chamber only when the sensor senses the medication pill in the rotatable chamber. The sensor can further be configured to detect when the rotatable chamber includes more than one medication pill and execute appropriate actions which may include preventing the rotatable chamber from rotating to dispense the pills, providing instructions to the patient to save the additional pill(s) for the next dosing time and skipping dispensing a pill in the next dosing time, etc.

The dispenser portion 150 can further include a biometric sensor 152 such as a fingerprint scanner or other suitable biometric sensor as understood by a person skilled in the pertinent art. The biometric sensor 152 can be positioned on a sidewall 168 of the dispenser portion 150. Additionally, or alternatively, the dispenser portion 150 can be configured to communicate with an external computing device having a biometric sensor (e.g. a patient's smart phone), biometric identification of the patient can be performed by the external computing device, and the verification can be communicated to the dispenser portion 150. The biometric sensor 152 can be configured to authenticate a patient or other authorized user of the pill dispenser assembly 100. The biometric sensor 152 can also be configured to cause the pill dispenser assembly 100 to dispense medication when an authorized patient or other user is authenticated. The biometric sensor 152 can be otherwise configured to provide security to ensure a patient-prescription-medication match.

The pill dispenser assembly 100 can further include a status indicator 166. Preferably the status indicator 166 is integral to the dispenser portion 150 and in communication with electrical circuitry of the dispenser portion 150. The status indicator 166 can include a visual indicator as illustrated. The visual indicator can be configured to change colors to indicate a status of the pill dispenser. Additionally, or alternatively, the visual indicator can include a display screen configured to display text and/or an icon indicating the status of the pill dispenser assembly 100. Additionally, or alternatively, the status indicator 166 can include a speaker configured to provide an auditory indication of status. The auditory indication of status can be in the form of beeps, melodic tones, and/or spoken language. Examples of status that can be indicated by the status indicator 166 can include prompt to patient to take medication, status of charge of battery in the dispenser portion 150, location of the medication (i.e. alarm and/or wireless beacon to help user/patient locate the pill dispenser assembly 100), quantity of medication remaining, a change in prescription dosage, a prompt to prepare cartridge for return/recycling, malfunction of the pill dispenser assembly 100, status of an upcoming prescription refill, attempt to be used by an unauthorized user, prompts to authenticate and authorize a new patient/user, attempt to dispense medication at an unauthorized time by an authorized patient/user, a history of medication dispensing, patient information, patient medical history, list of prescriptions being administered to patent, list of side effects of the prescribed medication, pharmacy contact information, prescribing physician contact information, customer support contact information for pill dispenser assembly, health insurance contact information, etc.

The pill dispenser assembly 100 can further include one or more medication sensors configured to monitor medication within the cartridge 130 and/or confirm dispensing of medication through the pill dispensing cavity 154.

Multiple pill dispenser assemblies 100 can be linked to a single patient profile so that a patient may have multiple pill dispenser assemblies 100 each configured to dispense medication according to a distinct prescription. Additionally, or alternatively, multiple cartridges 130 can be linked to a single dispenser portion 150, thereby allowing a user to interchange cartridges as needed.

FIG. 1C is an illustration of the cartridge 130 securing medication 142. The medication 142 is loaded into a primary cavity 139 of the cartridge 130. As illustrated, the medication 142 can be loaded into the primary cavity 139 prior to an angled barrier 136 being secured to a shell 131 of the cartridge 130. The cartridge 130 can be modified so that it can be loaded from the top or loaded after the angled barrier 136 are secured to the shell 131. For instance, a specialized funnel (not illustrated) can be configured to allow medication to be fed through the funnel through the angled barrier 136 into the primary cavity 139. The primary cavity 139 is sized and shaped to accommodate pill form medication. The primary cavity 139 can have an internal volume similar to that of known standard pill bottles. The internal volume of the primary cavity 139 can be bounded by the shell 131, the central tube 134, and the angled barrier 136.

The angled barrier 136 can be configured to allow loading of medication into the primary cavity 139 of the cartridge 130 and inhibit removal of medication 142 from the primary cavity 139. The angled barrier 136 can include disks with openings that can be moved into alignment to allow medication 142 to pass through the angled barrier 136 and can be moved out of alignment to prevent removal of medication 142 from the primary cavity 139. The disks can be out of alignment when the cartridge is provided to a patient, and the angled barrier 136 can be configured such that the patient is unable to move the disks into alignment. The angled barrier 136 can include an opening through which a portion of the mating protrusion 156 is sized to extend. The dispensing portion 150 can include a motor to cause the portion of the mating protrusion 156 to rotate and thereby cause at least one of the disks of the angled barrier 136 to rotate so that the disks are in alignment to allow medication 142 to pass through the angled barrier 136 into a pill passage and/or movable cavity of the dispensing portion 150.

The pill dispenser assembly 100 is preferably cylindrical; however, the pill dispenser assembly 100 can be any suitable shape as understood by a person skilled in the pertinent art according to the teachings herein.

The shell 131 is preferably impact resistant.

The dispenser portion 150 preferably includes a rechargeable battery having a battery life of up to 30 days after a full charge.

The pill dispenser assembly 100 is preferably configured to dispense only one pill at a time.

The pill dispenser assembly 100 preferably incorporates mechanical and sensing defenses to tampering.

The pill dispenser assembly 100 can preferably be paired to a user device of a patient or and/or another authorized user. The user device can include a computing device such as a smart phone, tablet, personal computer, etc. Once paired, an application on the user device can onboard new cartridges, verifies dosing schedules and allows for dynamic prescription adjustment and titration, as specified by the provider.

The cartridge 130 can be sized, shaped, and otherwise configured to accommodate and dispense pills within a range of sizes and within a range of shapes such that pills of significantly differing sizes or significantly differing shapes can be accommodated and dispensed from differently configured cartridges 130. The dispensing portion 150 preferably can be configured to mate with differently configured cartridges 130 such that the dispensing portion need not be separately configured based on pill size or shape to the same extent as the cartridge 130. Preferably, the dispensing portion 150 can be configured to dispense pills for all currently prescribed pill shapes and sizes. Alternatively, the dispensing portion 150 can be configured to dispense pills for an overwhelming majority of the most common currently prescribed pill shapes and sizes. Alternatively, the dispensing portion 150 can be configured to dispense pills for all currently prescribed high risk medications. Alternatively, the dispensing portion 150 can be configured to dispense pills for an overwhelming majority of currently prescribed high risk medications. A collection of cartridges 130 of differing configurations can likewise be collectively configured to accommodate and dispense pills in the aforementioned ranges of pill size and shape.

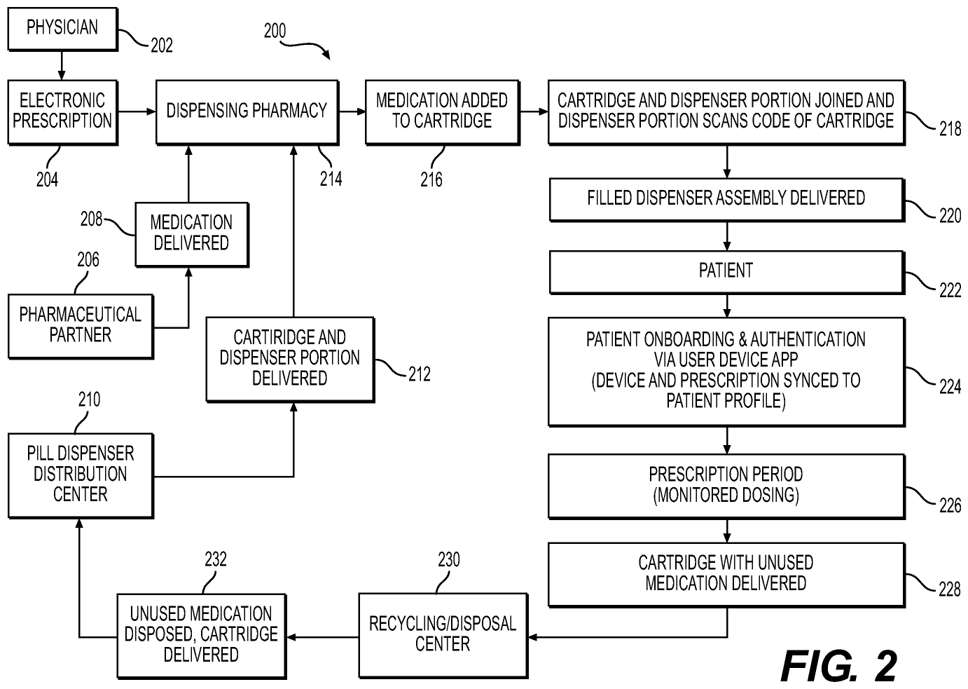

FIG. 2 is an illustration of a first medication distribution model 200 to provide medication to a new patient 222. The model 200 illustrates the interaction of a physician 202, a dispensing pharmacy 214, a pharmaceutical partner 206, a pill dispenser distribution center 210, a recycling/disposal center 230, and the patient 222. The model 200 can provide coverage of medication dispensing, security, dosing, adherence, tracking, and disposal.

At step 204, the physician 202 can provide an electronic prescription to the dispensing pharmacy 214. The electronic prescription 204 can communicate what medication and dosage schedule is prescribed to the patient 222 similar to existing prescription practices.

At step 208, the pharmaceutical partner 206 can deliver the prescribed medications to the dispensing pharmacy 214 according to the electronic prescription provided in step 204 similar to existing prescription practices. The pharmaceutical partner 206 may not deliver directly to the patient pharmacy but rather to a central pharmacy that then delivers to the patient pharmacy similar to current practices. The pharmaceutical partner 206 therefore may represent both the central pharmacy and the patient pharmacies.

At step 212, the pill dispenser distribution center 210 can deliver a cartridge and dispenser portion (similar to the cartridge 130 and dispenser portion 150 illustrated in FIGS. 1A-1C) to the dispensing pharmacy 214.

At step 216, the dispensing pharmacy 214 can add the medication received from the pharmaceutical partner 206 at step 208 to the cartridge received from the pill dispenser distribution center 210 at step 212.

At step 218, the dispensing pharmacy 214 can join the dispenser portion to the cartridge. The dispenser portion can scan a code of the cartridge at step 218 to thereby provide instructions to the dispenser portion so that medication can be dispensed according to the electronic prescription and the dispenser portion can otherwise perform various functions of the dispensing device according to the prescription.

At step 220, the filled dispenser assembly can be delivered to the patient 222.

At step 224, the patient can interact with the filled dispenser assembly and/or an application on a user device to onboard and authenticate the user. The filled dispenser assembly and the electronic prescription can be synchronized to a patient profile. The filled dispenser assembly can further be paired to the user device such that the dispenser assembly and user device are capable of communicating, preferably wirelessly.

At step 226, the patient can interact with the dispenser assembly to receive medication according to the electronic prescription provided at step 204. Although not illustrated, during the prescription period, updates in the prescription including a change in number and/or frequency of dispensing schedule of pills in the dispenser assembly can be pushed from the physician 202 to the dispenser assembly via a network (e.g. internet) and the patient's user device. The dispenser assembly can be configured such that when a treatment regime is complete, the dispenser assembly ceases all further dispensing of medication from the present cartridge.

At step 228, the prescription period is completed, and the patient 222 can deliver the unused medication to the recycling/disposal center. The dispenser assembly can be configured such that when the treatment regime is complete, the dispenser portion prompts for the cartridge to be returned to the recycling/disposal center 230 for safe disposal of unused medication. The patient 222 can retain the dispenser portion of the pill dispenser assembly for use with a new cartridge.

At step 232, the recycling/disposal center can dispose of unused medication and can deliver the empty cartridge to the pill dispenser distribution center 210.

As an alternative to the illustrated model 200, the recycling/disposal center 230 associated steps 228 and 232 can be omitted. In this alternative model, the patient 222 may be responsible for disposing of the cartridge and unused medication by other means (e.g. throwing in trash) similar to current common practices related to pill bottle and unused medication disposal. Examples which include the recycling/disposal center 230 are preferred to avoid unsafe disposal of unused medication.

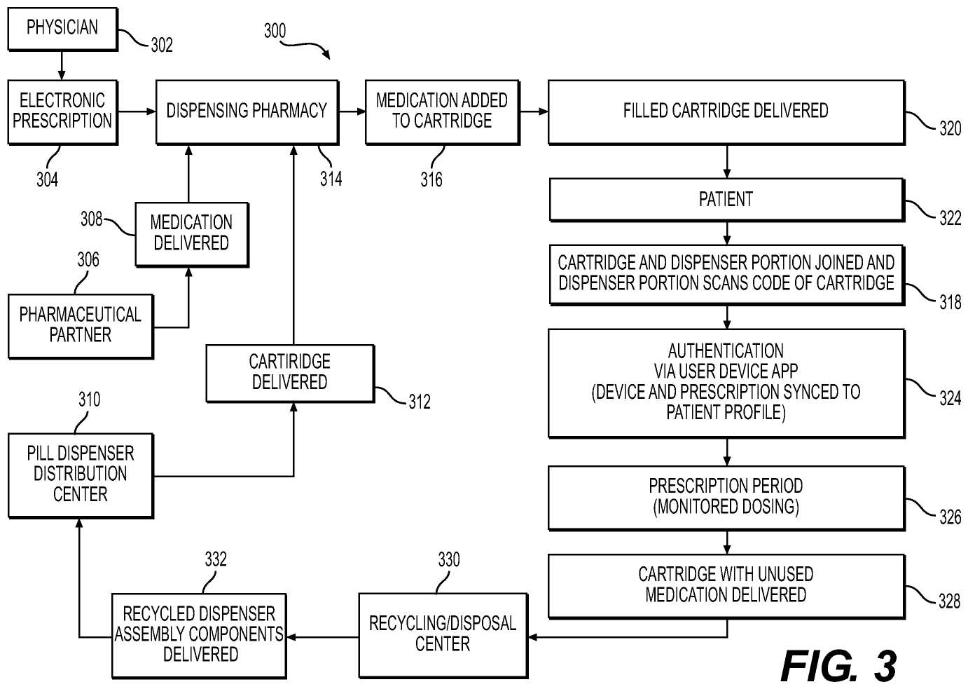

FIG. 3 is an illustration of a second medication distribution model 300 to provide medication to an existing patient 322. The model 300 illustrates the interaction of a physician 302, a dispensing pharmacy 314, a pharmaceutical partner 306, a pill dispenser distribution center 310, a recycling/disposal center 330, and the patient 322 which can be the same or different from the corresponding parties 202, 214, 206, 210, 230, 222 of the first medication distribution model 200. The second medication distribution model 300 can provide coverage of medication dispensing, security, dosing, adherence, tracking, and disposal.

At step 304, the physician 302 can provide an electronic prescription to the dispensing pharmacy 314. The electronic prescription can communicate what medication and dosage schedule is prescribed to the patient 322 similar to existing prescription practices.

At step 308, the pharmaceutical partner 306 can deliver the prescribed medications to the dispensing pharmacy 314 according to the electronic prescription provided in step 304 similar to existing prescription practices. The pharmaceutical partner 306 may not deliver directly to the patient pharmacy but rather to a central pharmacy that then delivers to the patient pharmacy similar to current practices. The pharmaceutical partner 306 therefore may represent both the central pharmacy and the patient pharmacies.

At step 312, the pill dispenser distribution center 310 can deliver a cartridge (similar to the cartridge 130 illustrated in FIGS. 1A-1C) to the dispensing pharmacy 314.

At step 316, the dispensing pharmacy 314 can add the medication received from the pharmaceutical partner 306 at step 308 to the cartridge received from the pill dispenser distribution center 310 at step 312.

At step 320, the filled cartridge can be delivered to the patient 322.

At step 318, the patient 322 can join a dispenser portion that has been previously delivered to the patent (e.g. for a previous prescription) to the cartridge. The dispenser portion can scan a code of the cartridge to thereby provide instructions to the dispenser portion so that medication can be dispensed according to the electronic prescription and the dispenser portion can otherwise perform various functions of the dispensing device according to the prescription.

At step 324, the patient can interact with the filled dispenser assembly and/or an application on a user device to onboard and authenticate the user. The filled dispenser assembly and the electronic prescription can be synchronized to a patient profile.

At step 326, the patient can interact with the dispenser assembly to receive medication according to the electronic prescription provided at step 304. Although not illustrated, during the prescription period, updates in the prescription including a change in number and/or frequency of dispensing schedule of pills in the dispenser assembly can be pushed from the physician 302 to the dispenser assembly via a network (e.g. internet) and the patient's user device. The dispenser assembly can be configured such that when a treatment regime is complete, the dispenser assembly ceases all further dispensing of medication from the present cartridge.

At step 328, the prescription period is completed, and the patient 322 can deliver the unused medication to the recycling/disposal center. The dispenser assembly can be configured such that when the treatment regime is complete, the dispenser portion prompts for the cartridge to be returned to the recycling/disposal center 330 for safe disposal of unused medication.

At step 332, the recycling/disposal center can dispose of unused medication and can deliver the empty cartridge to the pill dispenser distribution center 310.

As an alternative to the illustrated model 300, the recycling/disposal center 330 associated steps 328 and 332 can be omitted. In this alternative model, the patient 322 may be responsible for disposing of the cartridge and unused medication by other means (e.g. throwing in trash) similar to current common practices related to pill bottle and unused medication disposal. Examples which include the recycling/disposal center 330 are preferred to avoid unsafe disposal of unused medication.

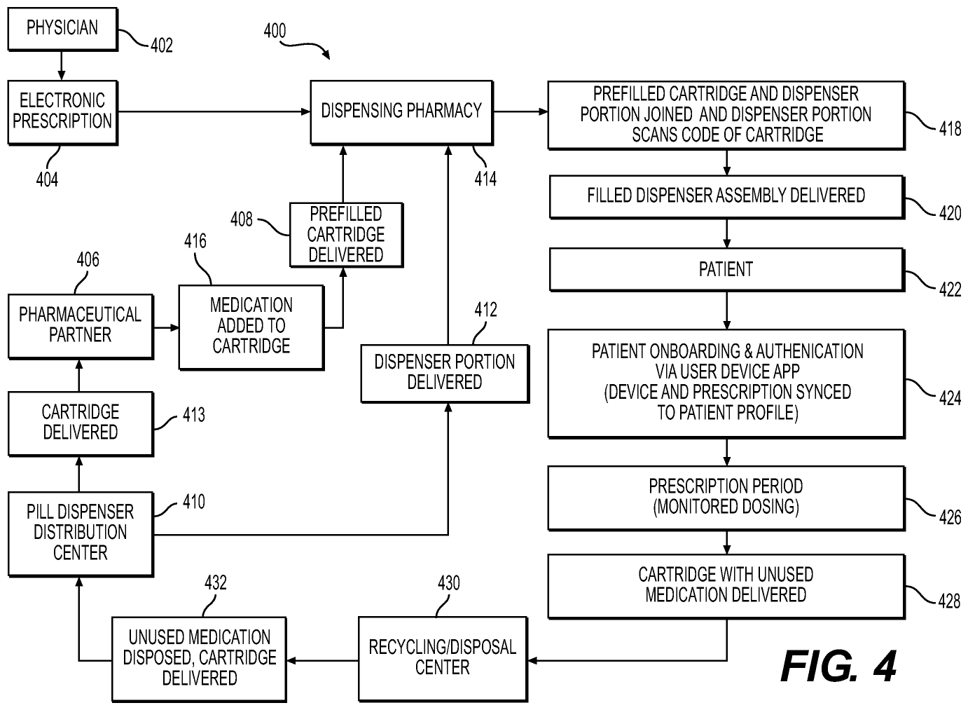

FIG. 4 is an illustration of a third medication distribution model 400 to provide medication to a new patient 422. The model 400 illustrates the interaction of a physician 402, a dispensing pharmacy 414, a pharmaceutical partner 406, a pill dispenser distribution center 410, a recycling/disposal center 430, and the patient 422 which can be the same or different from the corresponding parties 202, 214, 206, 210, 230, 222, 302, 314, 306, 310, 330, 322 of the first medication distribution model 200 and/or the second medication distribution model 300. The third medication distribution model 400 can provide coverage of medication dispensing, security, dosing, adherence, tracking, and disposal.

At step 404, the physician 402 can provide an electronic prescription to the dispensing pharmacy 414. The electronic prescription can communicate what medication and dosage schedule is prescribed to the patient 422 similar to existing prescription practices.

At step 413, a cartridge is delivered from the pill dispenser distribution center 410 to the pharmaceutical partner 406. The cartridge can be delivered to the pharmaceutical partner 406 upon a request from the pharmaceutical partner to the pill distribution center 410.

At step 416, the pharmaceutical partner 406 can add medication to the cartridge received from the pill dispenser distribution center 410 at step 413.

At step 408, the pharmaceutical partner 406 can deliver the prefilled cartridge to the dispensing pharmacy 414 according to the electronic prescription provided in step 404.

At step 412, the pill dispenser distribution center 410 can deliver a dispenser portion (similar to the dispenser portion 150 illustrated in FIGS. 1A-1B) to the dispensing pharmacy 414.

At step 418, the dispensing pharmacy 414 can join the dispenser portion to the cartridge. The dispenser portion can scan a code of the cartridge to thereby provide instructions to the dispenser portion so that medication can be dispensed according to the electronic prescription and the dispenser portion can otherwise perform various functions of the dispensing device according to the prescription.

At step 420, the filled dispenser assembly can be delivered to the patient 422.

At step 424, the patient can interact with the filled dispenser assembly and/or an application on a user device to onboard and authenticate the user. The filled dispenser assembly and the electronic prescription can be synchronized to a patient profile. The filled dispenser assembly can further be paired to the user device such that the dispenser assembly and user device are capable of communicating, preferably wirelessly.

At step 426, the patient can interact with the dispenser assembly to receive medication according to the electronic prescription provided at step 404. Although not illustrated, during the prescription period 426, updates in the prescription including a change in number and/or frequency of dispensing schedule of pills in the dispenser assembly can be pushed from the physician 402 to the dispenser assembly via a network (e.g. internet) and the patient's user device. The dispenser assembly can be configured such that when a treatment regime is complete, the dispenser assembly ceases all further dispensing of medication from the present cartridge.

At step 428, the prescription period is completed, and the patient 422 can deliver the unused medication to the recycling/disposal center. The dispenser assembly can be configured such that when the treatment regime is complete, the dispenser portion prompts for the cartridge to be returned to the recycling/disposal center 430 for safe disposal of unused medication. The patient 422 can retain the dispenser portion of the pill dispenser assembly for use with a new cartridge.

At step 432, the recycling/disposal center can dispose of unused medication and can deliver the empty cartridge to the pill dispenser distribution center 410.

As an alternative to the illustrated model 400, the recycling/disposal center 430 associated steps 428 and 432 can be omitted. In this alternative model, the patient 422 may be responsible for disposing of the cartridge and unused medication by other means (e.g. throwing in trash) similar to current common practices related to pill bottle and unused medication disposal. Examples which include the recycling/disposal center 430 are preferred to avoid unsafe disposal of unused medication.

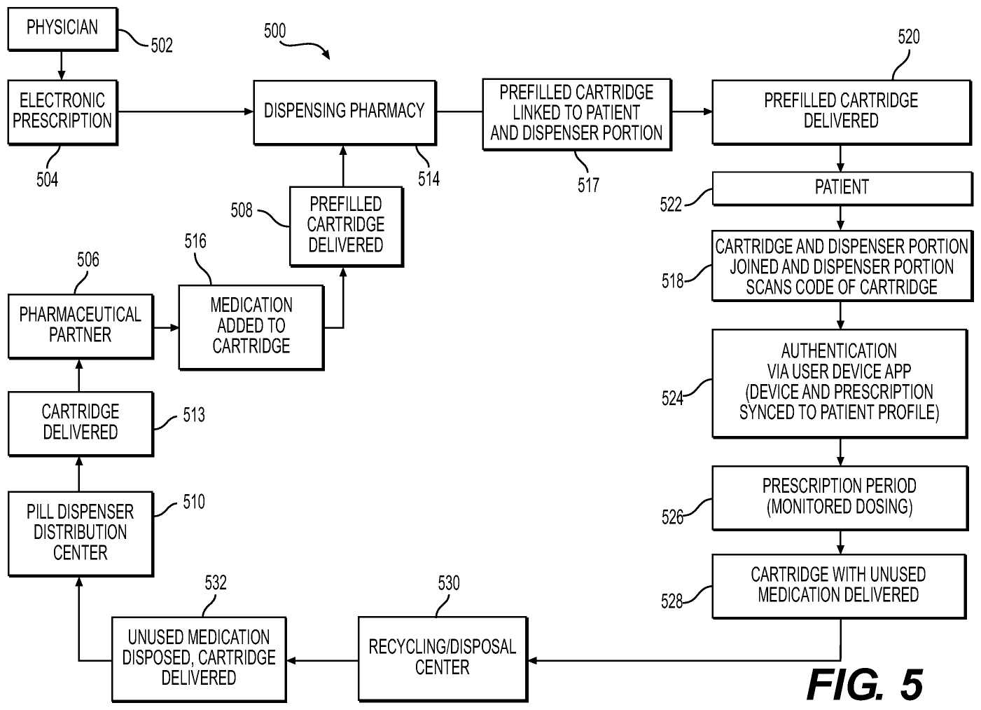

FIG. 5 is an illustration of a fourth medication distribution model 500 to provide medication to an existing patient. The model 500 illustrates the interaction of a physician 502, a dispensing pharmacy 514, a pharmaceutical partner 506, a pill dispenser distribution center 510, a recycling/disposal center 530, and the patient 522 which can be the same or different from the corresponding parties 202, 214, 206, 210, 230, 222, 302, 314, 306, 310, 330, 322, 402, 414, 406, 410, 430, 422 of the first medication distribution model 200, the second medication distribution model 300, and/or the third medication distribution model 400. The fourth medication distribution model 500 can provide coverage of medication dispensing, security, dosing, adherence, tracking, and disposal.

At step 504, the physician 502 can provide an electronic prescription to the dispensing pharmacy 514. The electronic prescription can communicate what medication and dosage schedule is prescribed to the patient 522 similar to existing prescription practices.

At step 513, a cartridge is delivered from the pill dispenser distribution center 510 to the pharmaceutical partner 506. The cartridge can be delivered to the pharmaceutical partner 506 upon a request from the pharmaceutical partner to the pill distribution center 510.

At step 516, the pharmaceutical partner 506 can add medication to the cartridge received from the pill dispenser distribution center 510 at step 513.

At step 508, the pharmaceutical partner 506 can deliver the prefilled cartridge to the dispensing pharmacy 514 according to the electronic prescription provided in step 504.

At step 517, the dispensing pharmacy 514 can link the prefilled cartridge to the patient and to a dispenser portion associated with, and in the possession of the patient (e.g. from a previous prescription cycle).

At step 520, the prefilled cartridge can be delivered to the patient 522.

At step 518, the patient 522 can join the dispenser portion to the cartridge. The dispenser portion can scan a code of the cartridge to thereby provide instructions to the dispenser portion so that medication can be dispensed according to the electronic prescription and the dispenser portion can otherwise perform various functions of the dispensing device according to the prescription.

At step 524, the patient can interact with the filled dispenser assembly and/or an application on a user device to authenticate the user. The filled dispenser assembly and the electronic prescription can be synchronized to a patient profile. The filled dispenser assembly can further be paired to the user device such that the dispenser assembly and user device are capable of communicating, preferably wirelessly.

At step 526, the patient can interact with the dispenser assembly to receive medication according to the electronic prescription provided at step 504. Although not illustrated, during the prescription period, updates in the prescription including a change in number and/or frequency of dispensing schedule of pills in the dispenser assembly can be pushed from the physician 502 to the dispenser assembly via a network (e.g. internet) and the patient's user device. The dispenser assembly can be configured such that when a treatment regime is complete, the dispenser assembly ceases all further dispensing of medication from the present cartridge.

At step 528, the prescription period is completed, and the patient 522 can deliver the unused medication to the recycling/disposal center. The dispenser assembly can be configured such that when the treatment regime is complete, the dispenser portion prompts for the cartridge to be returned to the recycling/disposal center 530 for safe disposal of unused medication. The patient 522 can retain the dispenser portion of the pill dispenser assembly for use with a new cartridge.

At step 532, the recycling/disposal center can dispose of unused medication and can deliver the empty cartridge to the pill dispenser distribution center 510.

As an alternative to the illustrated model 500, the recycling/disposal center 530 associated steps 528 and 532 can be omitted. In this alternative model, the patient 522 may be responsible for disposing of the cartridge and unused medication by other means (e.g. throwing in trash) similar to current common practices related to pill bottle and unused medication disposal. Examples which include the recycling/disposal center 530 are preferred to avoid unsafe disposal of unused medication.

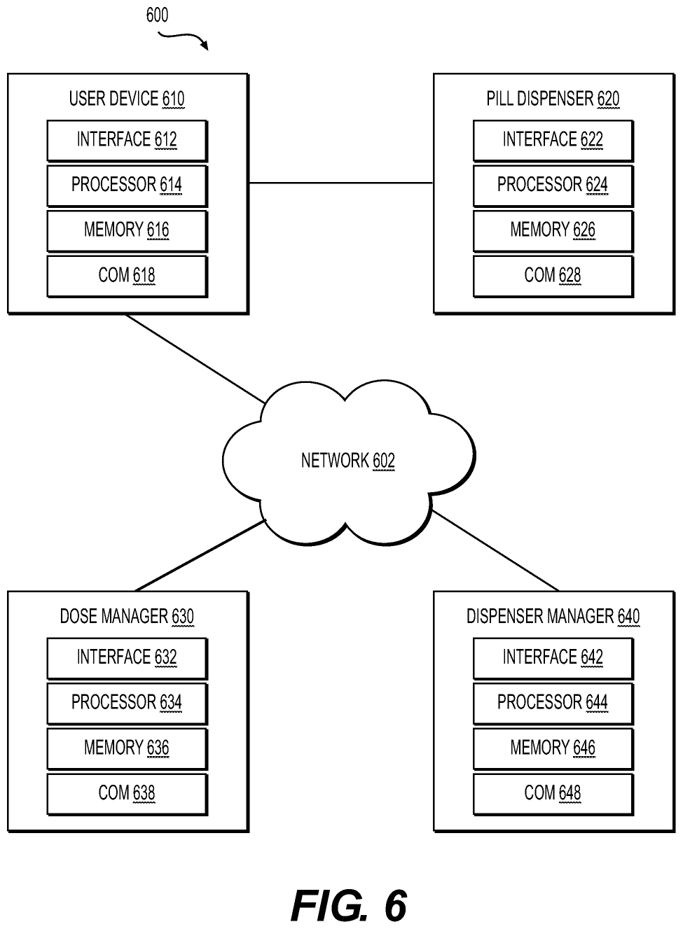

FIG. 6 is an illustration of a prescription treatment system 600 including a user device 610, a pill dispenser 620, a dose manager 630, and a dispenser manager 640, each of which includes a computing system having a respective interface 612, 622, 632, 642, processor 614, 624, 634, 644, memory 616, 626, 636, 646, and communicator 618, 628, 638, 648. The user device 610, dose manager 630, and dispenser manager 640 can each be in communication with a network 602 via the respective communicator 618, 638, 648. For the sake of simplicity, the network 602 is illustrated as being centralized and equally accessible to each of the user device 610, dose manager 630, and dispenser manager 640; however multiple alternative network topologies are possible as understood by a person skilled in the pertinent art according to the teachings herein.

As illustrated, the communicator 628 of the pill dispenser 620 is configured to communicate locally with the user device 610. This is assuming the pill dispenser 620 is in the vicinity of the user device 610 for the purposes of illustration, and it is to be understood that direct communication between the user device 610 and the pill dispenser 620 may break when the pill dispenser 620 is out of range of the user device 610. For instance, the user device 610 and pill dispenser 620 may be configured to communicate via a wired connection, or more preferably through a local wireless network (e.g. Bluetooth). Communications between the pill dispenser 620 to the remote dose manager 630, dispenser manager 640, and other network devices can be relayed through the user device 610. Additionally, or alternatively, the communicator 628 of the pill dispenser 620 can be configured to communicate directly to the network 602, in which case the pill dispenser 620 may communicate to the user device 610, dose manager 630, and/or dispenser manager 640 via the network 602.

The user device 610 can be configured to provide an interface for an end user such as a patient to the treatment system 600. The pill dispenser 620 can be configured to dispense pills and perform other “smart” dispenser functions such as described in relation to the pill dispenser assembly 100 illustrated in FIGS. 1A-1B. The dose manager 630 can be configured to provide an interface for a physician to interface with the prescription treatment system 600. The dispenser manager 640 can be configured to provide an interface for a pill dispenser manager to interface with the prescription treatment system 600.

The user device 610 can include a smartphone, tablet, personal computer, etc. The interface 612 can include a touch screen, display, keyboard, mouse, etc. to provide the user ability to interact with the user device 610. The processor 614 can be configured to execute instructions stored on the memory 616 (e.g. app). The app can be configured allow the end user to manage patient-facing aspects of the patients profiles. For instance, the app can be configured to onboard new cartridges, verify dosing schedules, pair with the pill dispenser 620, push notifications related to dynamic prescription adjustment and titration as specified by a provider/physician, etc.

The pill dispenser 620 is preferably configured similarly to the dispenser portion 150 of the pill dispenser assembly 100 illustrated in FIGS. 1A-1B. The pill dispenser 620 can be alternatively configured as understood by a person skilled in the pertinent art according to the teachings herein. For instance, the pill dispenser 620 may have a non-separable cartridge and dispenser portion.

The pill dispenser 620 can include sensors (e.g. biometric sensor 152 and dose monitoring sensors) similar to those of the pill dispensing assembly 100 illustrated in FIGS. 1A-1B. The pill dispenser 620 can be configured to secure and dispense medication similar to the pill dispensing assembly 100 similar to the pill dispensing assembly 100 illustrated in FIGS. 1A-1B. The interface 622 of the pill dispenser 620 can include components to allow the end user to interact with the pill dispenser 620. For instance, the interface 622 can include a biometric sensor, a pill dispensing cavity, and/or a status indicator configured similarly to corresponding structures 152, 154, 166 of the pill dispensing assembly 100 illustrated in FIGS. 1A-1B.

The processor 624 can be configured to execute instructions stored on the memory 626. The instructions can cause the processor to pair the pill dispenser 620 to the user device 610, associate the pill dispenser 620 to a patient profile, receive prescription information, dispense pills from the pill dispensing cavity based at least in part on the prescription information, determine a quantity of pills present in the pill dispenser 620, determine a quantity of pills dispensed by the pill dispenser, communicate to the user device 610, perform functions as described in relation to the pill dispensing assembly 100 illustrated in FIGS. 1A-1B, perform functions as described in relation to the dispensing assembly of the models 200, 300, 400, 500 illustrated in FIGS. 2-5, additional functions as understood by a person skilled in the pertinent art, and any sub-combinations thereof. If the communicator 628 of the pill dispenser 620 is configured to communicate with the network 602, the pill dispenser 620 can further be configured to communicate to the dose manager 630 and the dispenser manager 640 via the network 602.

The network communicator 628 of the pill dispenser 620 can be configured to communicate directly with the network 602, communicate directly with a computing device without requiring the network 602, and/or to communicate to the network 602 via another computing device. For instance, a dispensing pharmacy can provide prescription information to the pill dispenser 620 via direct communication between the dose manager 630 and the pill dispenser 620 while the pill dispenser is at the dispensing pharmacy. As another example, the pill dispenser 620 can receive and/or push information to the dose manager 630 and or dispenser manager via a direct communication to the user device 610 which is then in communication with the network 602. As another example, a pill dispenser distribution center can provide an initial configuration of the pill dispenser 620 via a direct communication between the pill dispenser 620 and the dispenser manager 640 while the pill dispenser 620 is at the pill dispenser distribution center.

The dose manager 630 preferably includes a HIPPA compliant web portal configured to allow doctors to manage patients and prescriptions, view patient compliance in real-time, and initiate actions. The does manager 630 can be configured to collect and aggregate data from many patients, which can thereby provide the opportunity to facilitate drug use and efficacy studies. The dose manager 630 can be configured to push updated prescription data to the pill dispenser 620, receive dose administration information from the pill dispenser 620, push notifications to the user device 610, receive patient communications/profile updates from the user device 610, perform other functions related to managing patient prescriptions as described in relation to models 200, 300, 400, 500 illustrated in FIGS. 2-5, additional functions as understood by a person skilled in the pertinent art, and any sub-combinations thereof.

The dispenser manager 640 can collect data related to the life cycle of the pill dispenser 620, push commands to the pill dispenser 620, push notifications to the user device 610, push notifications to the dose manager 630, perform other functions related to managing the pill dispenser 620 as described in relation to models 200, 300, 400, 500 illustrated in FIGS. 2-5, additional functions as understood by a person skilled in the pertinent art, and any sub-combinations thereof.

The descriptions contained herein are examples of embodiments of the invention and are not intended in any way to limit the scope of the invention. As described herein, the invention contemplates many variations and modifications of a pill dispensing device, a medication distribution model, a prescription treatment system, and methods associated therewith. Modifications and variations apparent to those having skilled in the pertinent art according to the teachings of this disclosure are intended to be within the scope of the claims which follow.

What is claimed is:

1. A pill dispenser assembly comprising:
a dispenser portion comprising a dispensing cavity, a movable cavity configured to move a medication pill into the dispensing cavity, a processor, and non-transitory computer readable memory in communication with the processor; and
a cartridge comprising a shell portion and an angled barrier extending within the shell portion,
wherein the shell portion and the angled barrier bound a primary cavity of the cartridge that is sized to receive a plurality of medication pills,
wherein the cartridge is configured to couple to the dispenser portion, and
wherein the cartridge is configured to secure the plurality of medication pills from removal from the primary cavity when the cartridge is decoupled from the dispenser portion.

2. The pill dispenser assembly of claim 1, wherein the non-transitory computer readable memory includes instructions thereon, such that when the cartridge is coupled to the dispenser portion and the instructions are executed by the processor, the instructions cause the pill dispenser assembly to:
authenticate a user of the pill dispenser assembly, and
actuate, at least in part in response to authentication of the user, the dispenser portion to move the movable cavity and dispense the medication pill into the dispensing cavity such that the medication pill is removable from the pill dispenser assembly.

3. The pill dispenser assembly of claim 2,
wherein the instructions further comprise prescription information of the user, and
wherein the processor is configured to actuate the dispenser portion to dispense the medication pill based at least in part on the prescription information.

4. The pill dispenser assembly of claim 3,
wherein the dispenser portion further comprises communication circuitry configured to communicate to an external computing device external to the pill dispenser assembly, and
wherein the instructions, when executed by the processor, further cause the processor to receive the prescription information from the external computing device.

5. The pill dispenser assembly of claim 1,
wherein the dispenser portion further comprises a biometric sensor, and
wherein the dispenser portion is configured to actuate to dispense the medication pill in response to an authentication signal provided from the biometric sensor.

6. The pill dispenser assembly of claim 1, wherein the dispenser portion further comprises a mating protrusion configured to abut the angled barrier of the cartridge when the cartridge is coupled to the dispenser portion.

7. The pill dispenser assembly of claim 6,
wherein the dispenser portion further comprises a central opening in the mating protrusion, and
wherein the cartridge further comprises a central tube extending within the shell portion such that when the mating protrusion is inserted into the opening of the shell portion the central opening and the central tube are aligned on a longitudinal axis of the pill dispenser assembly.

8. The pill dispenser assembly of claim 6,
wherein the mating protrusion further comprises an off-axis opening that is positioned radially offset from a longitudinal axis of the pill dispenser assembly, and
wherein the dispenser portion further comprises a pill passage sized to allow the medication pill to pass into the off-axis opening, through the pill passage, and out of the dispensing cavity.

9. The pill dispenser assembly of claim 1, wherein the dispenser portion further comprises a rechargeable battery and inductive charger.

10. The pill dispenser assembly of claim 1, wherein the dispenser portion further comprises a status indicator configured to provide a visual indication of a status of the pill dispenser assembly.

11. The pill dispenser assembly of claim 10, wherein the status indicator comprises a color-changing light ring circumscribing the dispenser portion.

12. The pill dispenser assembly of claim 1,
wherein the angled barrier includes disks each with a respective opening such that at least one of the disks is rotatable to move the openings into alignment and rotatable to move the openings out of alignment.

13. The pill dispenser assembly of claim 12,
wherein the dispenser portion further comprises a mating protrusion configured to abut the angled barrier of the cartridge when the cartridge is coupled to the dispenser portion,
wherein the angled barrier further includes an opening through which a portion of the mating protrusion is sized to extend,
wherein the dispensing portion includes a motor configured to cause the portion of the mating protrusion to rotate and thereby cause the at least one of the disks to rotate to move the openings into alignment and/or to move the openings out of alignment.

14. A user device comprising:
a user interface;
one or more transceivers;
one or more processors; and
non-transitory computer readable medium with instructions thereon, that when executed by the processor, cause the user device to:
communicate, via the one or more transceivers, to a pill dispenser;
display, on the user interface, a dosing schedule associated with the pill dispenser; and
transmit, via the one or more transceivers, to a dispenser manager configured to provide an interface to a pill dispenser manager, data related to a life cycle of the pill dispenser.

15. The user device of claim 14, wherein the instructions, when executed by the one or more processors, further cause the user device to:
onboard a cartridge of the pill dispenser.

16. The user device of claim 14, wherein the instructions, when executed by the one or more processors, further causes the user device to:
communicate, via the one or more transceivers, to a dose manager configured to provide an interface to a physician;
receive, via the one or more transceivers, from the dose manager, a prescription adjustment notification; and
transmit, via the one or more transceivers, to the pill dispenser, prescription adjustment instructions based at least in part on the prescription adjustment notification.

17. The user device of claim 14, wherein the instructions, when executed by the one or more processors, further causes the user device to:
receive, via the one or more transceivers, from the dispenser manager, an initial configuration of the pill dispenser; and
transmit, via the one or more transceivers, to the pill dispenser, configuration instructions based at least in part on the initial configuration.

18.-19. (canceled)

20. A dispenser manager, comprising:
a user interface;
one or more transceivers;
one or more processors; and
non-transitory computer readable medium with instructions thereon, that when executed by the processor, cause a user device to:
communicate, via the one or more transceivers, to a pill dispenser;
receive, via the one or more transceivers, from the pill dispenser, data related to a life cycle of the pill dispenser; and
transmit, via the one or more transceivers, to the pill dispenser, an initial configuration of the pill dispenser.
